(19) United States
(12) Plant Patent Application Publication
(10) Pub. No.: US 2002/0092045 P1
(43) Pub. Date: Jul. 11, 2002

(54) Rose plant named 'Taniripsa'

(75) Inventor: Hans-Jurgen Evers, Uetersen (DE)

Correspondence Address: Russell D. Orkin, 700 Koppers Building, 436 Seventh Avenue, Pittsburgh, PA, 15219-1818, US

(73) Assignee: Rosen Tantau Mathias Tantau Nachfolger

(21) Appl. No.: 09/758,721

(22) Filed: Jan. 11, 2001

(51) Int. Cl. A01H 5/00

(52) U.S. Cl. PLT/103

(57) ABSTRACT

The new cultivar is a variety of bed rose plant having an upright bushy growth habit. The white flowers are numerous. The plant exhibits good winter hardiness, resistance to most diseases, and good ground covering ability.

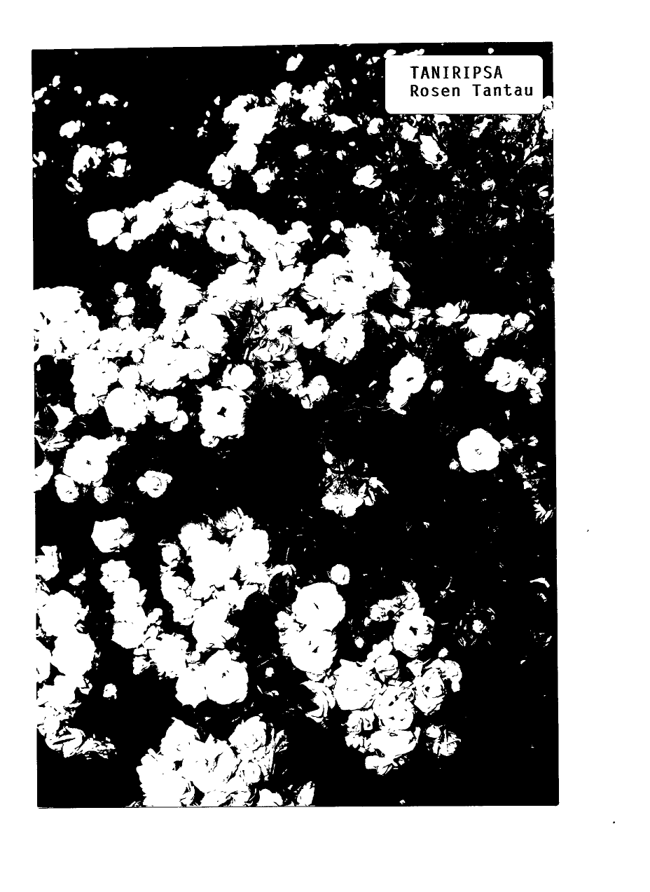

### BACKGROUND OF THE NEW PLANT

The present invention includes a new and distinct cultivar of Rosa l. known by the varietal name ‘Taniripsa’.

The plant was discovered in 1988 by the breeder in Ueterson, Germany in a selective breeding program designed to produce a robust bed rose for public and private gardens. The new plant is a selection from the cross of RT 83 350 (the female parent), a variety maintained proprietary by the assignee of the present application, and the unpatented variety ‘The Fairy’ (the male parent). The plant was first asexually reproduced in 1997 and can be propagated asexually on Rosa canina rootstock.

The variety is distinguishable by its very good winter hardiness, disease resistance, and good ground covering. Continued observations from the vegetative cuttings in Uetersen, Germany have confirmed that the distinguishing features of this new cultivar come true, remain stable, and are retained through successive propagations.

### DESCRIPTION OF THE DRAWING

The accompanying photographic drawing illustrates the new cultivar. Colors are described in part in reference to the R.H.S. Colour Chart of The Royal Horticultural Society of London.

### DESCRIPTION OF THE NEW PLANT

The following description sets forth the characteristics of the new cultivar.

The plant:

Botanical name.—Rosa l.

Size:

Height.—70 cm.

Width.—30 cm.

Habit: Bushy.

Growth vigor: Average.

Wood color: Green.

Thorns:

Color.—Bright brown.

Size.—0.8 cm.

Shape.—Elongated.

Location on stems and branches: Random arrangement.

Disease resistance: Robust to winter killing and fungi diseases.

Hardiness: Robust in winter.

### Foliage

Arrangement: Compound; unpaired pinnate.

Number of leaflets on typical stem leaves: 5.

Leaflets:

Size.—4 cm.

Color.—Green. Upper surface: Green. Lower surface: Green.

Shape.—Elliptical.

Texture.—Smooth.

Margin.—Serrated.

Petiole rachis:

Stipules.—Size: 4 cm. Shape: Elongated.

### Bud

Size: 3 cm.

Shape: Bowl-shaped.

Color: Pink changing to pure white during opening.

Sepals:

Color.—Green.

Texture.—Smooth.

Receptacle:

Color.—Greenish-white.

Shape.—Circular.

Texture.—Funnel-shaped.

### Flower

Borne (number in clusters): 6.

Size (diameter): 11 cm.

Form: Cluster.

Petals:

Number per flower: 41.

Color.—Upper surface: White Group 155B. Lower surface: White Group 155B.

Texture.—Smooth.

Size.—3.8 cm.

Shape.—Nearly circular.

Persistence.—High.

Lastingness.—High.

Fragrance: None.

Blooming period: Late June until November in middle European climate.

### Reproductive Organs

Stamens:

Number.—32.

Anther.—Length: 0.1 cm. Color: Brown.

Filament.—Length: 0.2 cm. Color: White.

Pistils:

Style.—White-green in color.

Stigma.—Yellow in color.

I claim:

1. A new and distinct variety of rose plant substantially as shown and described.
